(19) United States
(12) Patent Application Publication
(10) Pub. No.: US 2010/0049003 A1
(43) Pub. Date: Feb. 25, 2010

(54) EXPANDABLE SURGICAL SITE ACCESS SYSTEM

(76) Inventor: Elad I. Levy, Amherst, NY (US)

Correspondence Address: Jennifer Meredith;Lippes Mathias Wexler Friedman LLP, 665 Main Street, Suite 300, Buffalo, NY, 14203, US

(21) Appl. No.: 11/993,216

(22) PCT Filed: Jun. 23, 2006
(86) PCT No.: PCT/US06/24472

§ 371 Date: Dec. 19, 2007

Related U.S. Application Data
(60) Provisional application No. 60/693,446, filed on Jun. 24, 2005.

(51) Int. Cl. A61F 2/06; A61B 1/267

(52) U.S. Cl. 600/199; 623/1.2; 623/1.11

(57) ABSTRACT

An expandable surgical site access system and method for using the expandable surgical site access system to perform minimally invasive, percutaneous surgeries to access the spine or other bone structures, organs, or locations of the body is disclosed. In one embodiment, the expandable surgical site access system includes an elongated, expandable stent (20) that is particularly adapted to be deployed in a body during a surgical procedure to provide access to a surgical site (24) within the body. The stent (20) defines a working channel (26) through the body from a point of entry (28) to the surgical site (24).

The present application claims the filing benefit of U.S. Provisional Application Ser. No. 60/693,446, filed Jun. 24, 2005 and PCT/US2006/024472 the disclosures of which are hereby incorporated herein by reference in their entirety.

### FIELD OF THE INVENTION

The present invention relates generally to devices and methods for performing minimally invasive, percutaneous surgeries. More particularly, the present invention relates to a surgical instrument and method for providing access to a surgical site within a body.

### BACKGROUND OF THE INVENTION

Traditional surgical procedures often require a long incision, extensive muscle stripping, and prolonged retraction of tissues to access the desired surgical site as well as denervation and devascularization of surrounding tissue. This is particularly the case with spinal applications because of the need for access to locations deep within the body. Such surgical procedures can cause significant trauma to intervening tissues and potential damage to good tissue due to the amount and duration of tissue retraction, resulting in increased recovery time, permanent scarring, and pain that can be more severe than the pain that prompted the original surgical procedure. This is further exacerbated by the need to make a large incision so that the surgeon can properly view the areas inside the body that require attention.

Endoscopic, or minimally invasive, surgical techniques allow a surgical procedure to be performed on a patient's body through a smaller incision in the body and with less body tissue disruption. Endoscopic surgery typically utilizes a tubular structure known as a cannula (or portal) that is inserted into an incision in the body. A typical cannula is a fixed diameter tube, which a surgeon uses to hold the incision open and which serves as a conduit extending between the exterior of the body and the local area inside the body where the surgery is to be performed. Thus, cannulae can be used for visualization, instrument passage, and the like.

The typical cannula, however, presents at least two disadvantages. First, insertion of the cannula typically requires an incision. Although this incision is often relatively smaller than incisions made for surgical procedures performed without a cannula, there is still trauma to healthy tissue. Additionally, endoscopic surgical techniques may be limited by the size of the cannula because some surgical instruments, such as steerable surgical instruments used in posterior discectomies, are sometimes larger than the size of the opening defined by the cannula. Therefore, there is a need for a surgical site access system that can be inserted with minimal incision of tissue yet still provide an entrance opening and conduit sized for sufficient instrument passage and operation.

### SUMMARY OF THE INVENTION

The present invention overcomes the foregoing and other shortcomings and drawbacks of surgical site access systems heretofore known. While the invention will be described in connection with certain embodiments, it will be understood that the invention is not limited to these embodiments. On the contrary, the invention includes all alternatives, modifications and equivalents as may be included within the spirit and scope of the present invention.

The present invention is directed to a surgical site access system and method for performing minimally invasive, percutaneous surgeries to access the spine or other bone structures, organs or locations of the body. In one embodiment, the surgical site access system comprises an elongated, expandable stent that is particularly adapted to be deployed in a body during a surgical procedure to provide access to a surgical site within the body.

In accordance with one aspect of the present invention, the stent defines a working channel through the body from a point of entry to the surgical site. The working channel defines a passageway through which a surgeon may view the area of interest and pass surgical instruments and/or other devices (not shown) to the surgical site from outside the point of entry by providing a barrier against surrounding tissue, organs, bodily fluids and the like.

In one embodiment, the stent is a self-expanding stent that is deployed at the surgical site by a delivery catheter. The stent is delivered to the surgical site in a collapsed state on a distal end of the delivery catheter. Following deployment, the self-expanding stent expands from its compressed state outwardly to a greater first cross-sectional extent. In its deployed state, the stent is configured and constructed to resist inward pressure from soft tissue, organs and bodily fluids to maintain the open working channel from the point of entry to the surgical site.

In accordance with another aspect of the present invention, an inflation balloon may be inserted into the stent at the surgical site. The balloon is inflated within the stent to thereby expand the stent outwardly to a second cross-sectional extent greater than the first cross-sectional extent. The amount of expansion of the stent will depend on the configuration of the stent, the configuration and operation of the inflation balloon and the desired size and shape of the working channel defined by the expanded stent. The balloon is removed following expansion of the stent so that the expanded stent defines a working channel having a desired size and shape from the point of entry to the surgical site.

In accordance with another aspect of the present invention, the stent may be provided with an illumination system comprising one or more fiber optic rods or tubes or other suitable illumination devices. The illumination system may be configured to illuminate the working channel between the point of entry and the surgical site during a surgical procedure.

These and other objects and advantages of the present invention will be made apparent from the accompanying drawings and the description thereof.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the principles of the invention.

FIGS. 1-5 are diagrammatic views illustrating sequential steps for deploying a stent according to one embodiment of the present invention for providing access to a surgical site from a point of entry;

FIG. 6 is a diagrammatic side elevational view showing a portion of the stent of FIG. 2-5 being trimmed away generally near the point of entry;

FIG. 7 is a diagrammatic side elevational view showing a flexible stent according to one aspect of the present invention;

FIG. 8 is a diagrammatic side elevational view showing a stent according to another aspect of the present invention having a generally flared distal end;

FIG. 9 is a diagrammatic side elevational view showing a stent according to yet another aspect of the present invention having a generally rectangular cross-sectional profile along at least a portion thereof;

FIG. 10 is a cross-sectional view taken along line 10-10 of FIG. 9;

FIG. 11 is a diagrammatic side elevational view showing a stent according to still another aspect of the present invention having a generally cylindrical cross-sectional profile and a generally rectangular cross-sectional profile along different portions thereof;

FIG. 12 is a cross-sectional view of a stent according to another aspect of the present invention having a membrane secured thereto;

FIG. 13 is a diagrammatic perspective view showing an illumination system according to one aspect of the present invention associated with the stent of FIGS. 2-5;

FIG. 14 is a cross-sectional view of an alternative illumination system associated with the stent of FIGS. 2-5;

FIG. 15 is a cross-sectional view of a stent according to another aspect of the present invention impregnated with a bioactive material; and

FIG. 16 is a view similar to FIG. 15 showing a stent according to yet another aspect of the present invention having a bioactive material coated on an outer surface thereof.

### DETAILED DESCRIPTION OF THE PREFERRED EMBODIMENTS

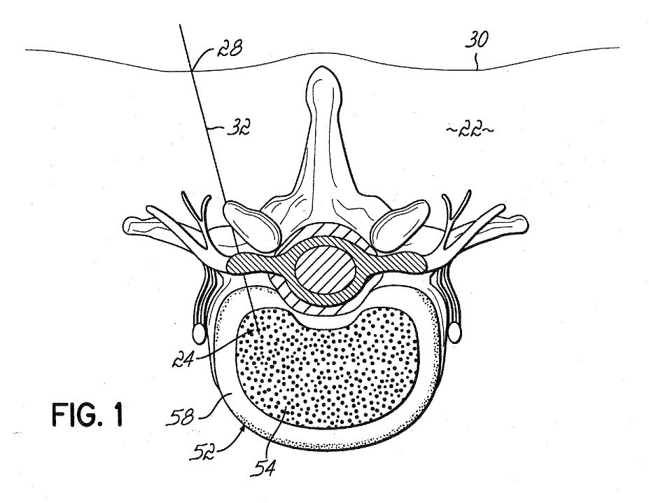

Referring now to the figures, and to FIGS. 1-5 in particular, an elongated, expandable stent 20 according to one embodiment of the present invention is shown. As will be described in greater detail below, stent 20 is particularly adapted to be deployed into a body 22 and expanded outwardly during a surgical procedure to provide access to a surgical site 24, possibly deep within the body, when the stent 20 is deployed and expanded. The stent 20 may be an integral component and is configured to maintain an open passage through soft tissue in the body for providing surgical access to a remote portion of the body during a surgical procedure.

In accordance with the principles of the present invention, the stent 20 defines a working channel 26 (see FIG. 13) through the body 22 from a point of entry 28 to the surgical site 24. The working channel 26 defines a passageway through which a surgeon may view the area of interest and pass surgical instruments and/or other devices (not shown) to the surgical site 24 from outside the point of entry 28 by providing a barrier against surrounding tissue, organs, bodily fluids and the like. It is understood that a working channel can be provided for any number of surgical procedures. The point of entry 28 may be a percutaneous opening made by piercing a patient's skin 30 with a needle (not shown) or a guide wire 32, by making a small incision in the patient's skin, or by any other minimally invasive approach well known to those skilled in the art.

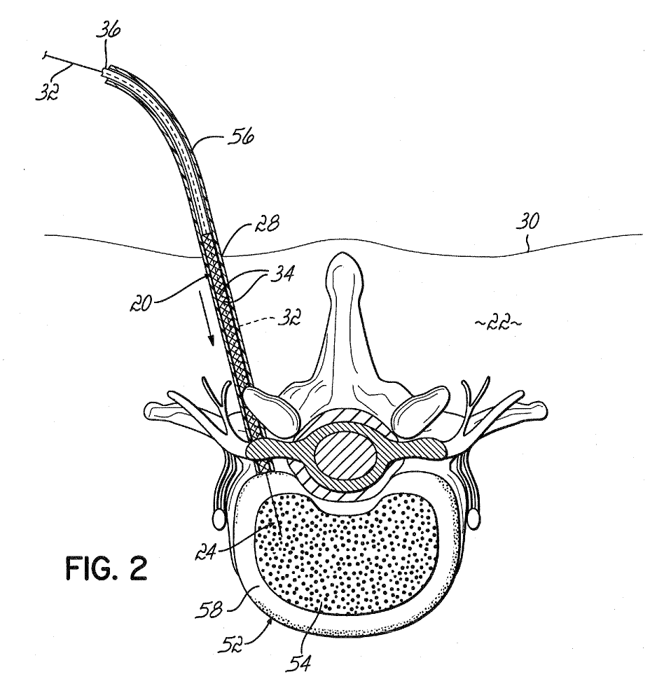

In one embodiment, the stent 20 may be a generally self-expanding stent comprising a mesh, lattice or other structure that has a shape memory configuration so that the stent 20 assumes an expanded, open lumen configuration when the stent 20 is allowed to seek its own natural configuration. In one embodiment, the stent 20 may comprise a set of braided wires 34 made of materials (for example, stainless steel wire, plastics, or Nitenol) and that can be set in the desired open, deployed configuration. The wires 34 can be braided into the desired configuration and then compressed for placement onto a delivery catheter 36 (see FIG. 2) as will be described in detail below. It will be appreciated that in other embodiments, the stent 20 may not be self-expanding and may comprise other mesh or non-mesh structures and/or be constructed of other metal or non-metal materials well known to those of ordinary skill in the art without departing from the spirit and scope of the present invention. It will also be appreciated that the stent 20 can be constructed of a material that allows for sequential dilation of the stent. The construction of the stent 20 may vary depending on the surgical procedure being performed, and its characteristics and construction may depend on such factors as the soft tissue, organ and bodily fluid barrier requirements, the desired size and shape of the working channel 26, and the location of the surgical site 24 within the body.

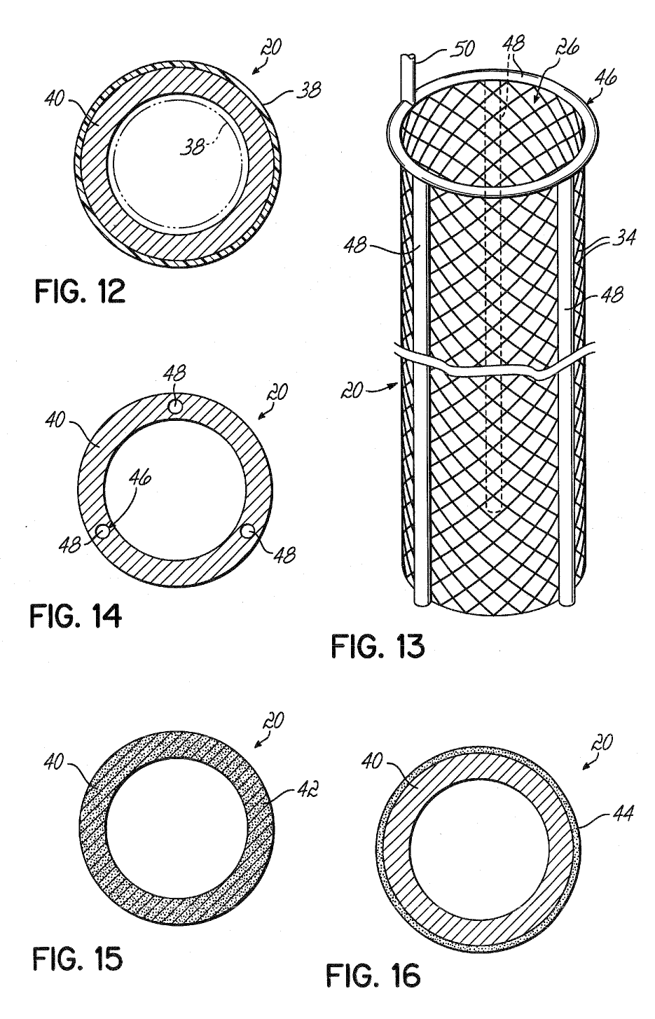

In one embodiment, as shown in FIG. 12, the stent 20 may be provided with a membrane 38 made of expanded PTFE. Other biocompatible materials are possible as well. The membrane 38 may be located around the outer perimeter of the stent wall 40 and, in one embodiment, may be finished over the ends of the braided wires 34 so that the wire ends are not exposed. Alternatively, as shown in FIG. 12, it is contemplated that the membrane 38 may be located on the inside perimeter of the stent wall 40. In yet another embodiment, both an inner and an outer membrane 38 may be secured to the stent wall 40 to completely envelope the braided wires 34. The membrane 38 provides a fluid barrier to minimize the inflow of bodily fluids through the wall 40 of the stent 20 and into the working channel 26 during a surgical procedure.

In one embodiment, the membrane 38 may include a bioactive material either impregnated into the membrane or provided as an outer coating thereon. The bioactive material may comprise one or more of an antibiotic, anti-inflammatory, pain alleviating and/or pro-thrombotic agent that leaches into the tissues surrounding the stent 20. The agent(s) may provide advantages in the post-operative period in terms of pain reduction, infection rates and bleeding, for example.

Alternatively, as shown in FIGS. 15 and 16, it is contemplated that the stent 20 may not include a membrane so that the wall 40 of the stent 20 directly contacts the surrounding tissue. In this embodiment, the stent wall 40 may be impregnated with one or more bioactive materials, as shown generally by numeral 42 in FIG. 15. In yet another embodiment, as shown in FIG. 16, the outer surface of the stent wall 40 may be coated with one or more bioactive materials, as shown generally by numeral 44 in FIG. 16, in a manner so that the agents leach into the surrounding tissue in contact with the stent wall 40.

Referring now to FIGS. 13 and 14, the stent 20 may be provided with an illumination system 46 comprising one or more fiber optic rods or tubes 48 or other suitable illumination devices. The fiber optic rods or tubes 48 may be made of any optical quality polymer, glass or other suitable material. The fiber optic rods or tubes 48 are coupled to an illuminating source (not shown) through a fiber optic feed 50. In one embodiment, as shown in FIG. 13, the fiber optic rods or tubes 48 are supported by the stent 20 and one or more of the rods or tubes 48 may extend generally axially the length of the stent 20 to illuminate the working channel 26 during the surgical procedure. Alternatively, as shown in FIG. 14, one or more of the fiber optic rods or tubes 48 may be integrated with the wall 40 of the stent 20 so as to form part of the stent wall structure. The fiber optic rods or tubes 48 are mounted to permit expansion of the stent 20 during its use to create the working channel 26 as described in greater detail below. It will be understood that other illuminating systems are possible as well.

The stent 20 of the present invention will now be described in connection with its use during a spinal discectomy procedure as shown in FIGS. 1-5. While the present invention will be described herein in connection with spinal surgery, it will be appreciated that the stent 20 of the present invention has broad uses in many varied surgical procedures requiring access through a body to a surgical site and is therefore not limited to spinal surgery per se.

In a first step of the procedure, as shown in FIG. 1, the guide wire 32 can be advanced through the skin 30 and soft tissue to the desired surgical site, such as the intervertebral disc 52. The guide wire 32 may be provided with a penetrating tip, and may also be provided with the capability to record EMG activity so as to minimize the risk of nerve root injury. The penetrating tip may be made of polyimide, PEEK, or suitable metals, polymers, or ceramics that allow the penetrating tip to be mated to the guide wire 32.

Preferably, a local anesthetic may be administered with an access needle (not shown) and a small incision of about 1 cm in length (the incision length may be varied depending on surgical procedure) is made in the skin 30 and underlying fascia to facilitate penetration of the guide wire 32 through the skin 30. The guide wire 32 may be advanced from the point of entry 28 to the surgical site 24 under fluoroscopy, direct visual guidance or any other suitable guidance method. After the guide wire 32 reaches its target, such as the intervertebral disc 52, the wire 32 may be advanced into the disc space 54 for access location as shown in FIG. 1.

In one embodiment, as shown in FIG. 2, the stent 20 is releasably mounted on the delivery catheter 36. The delivery catheter 36 may be made of polyesters, polybutylenes, polyamides, elastomers or the like. The stent 20 is collapsed when a self-expanding stent is used on a distal end of the delivery catheter 36 and the stent 20 may be covered, in one embodiment, by an elongated retaining sheath 56. As used herein, the term “distal” is intended to refer to a location remote from the surgeon while the term “proximal” is intended to refer to a location closer to the surgeon. The delivery catheter 36 and the stent 20 are advanced along the guide wire 32 to the surgical site 24 where the stent 20 is to be deployed. The retaining sheath 56 is a retractable sheath that holds the stent 20 in a compressed configuration (when a self-expanding stent is used) until the sheath 56 is moved or retracted off of the stent 20 so that the stent 20 is no longer radially constrained and is released from the distal end of the delivery catheter 36. In one embodiment, the stent 20 may have an outer diameter in its compressed state of between about 3 mm and about 12 mm, although other outer diameters are possible as well for the stent 20 depending on the working channel requirements for a particular surgical procedure.

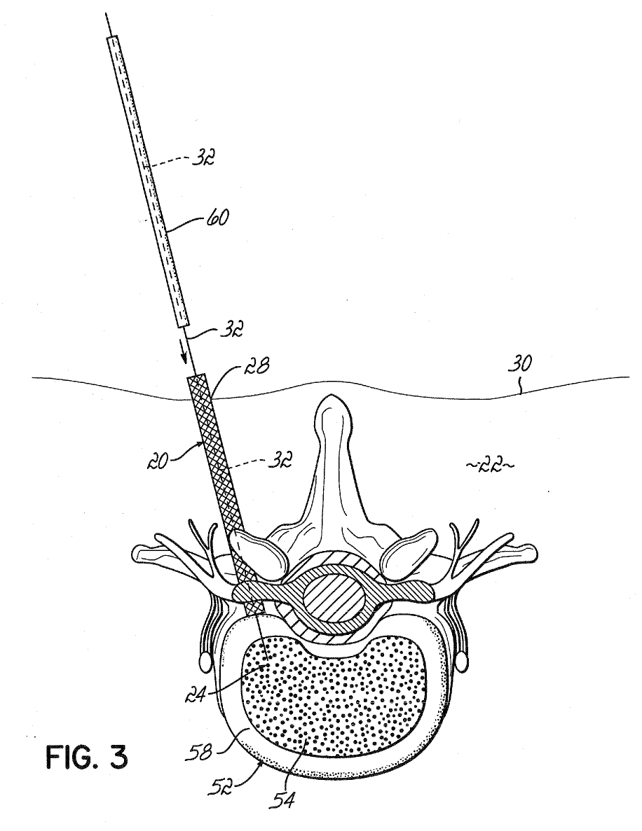

As shown in FIG. 3, when the delivery catheter 36 and retaining sheath 56 are removed, the stent 20 is deployed at the surgical site 24 and extends from outside of the point of entry 28 to the annulus 58 of the intervertebral disc 52. In the case of a self-expanding stent, the deployed stent 20 expands from its compressed state outwardly to a greater first cross-sectional extent. In its deployed state, the stent 20 is configured to resist inward pressure from soft tissue, organs and bodily fluids to maintain the open working channel 26 from the point of entry 28 to the surgical site 24. In one embodiment, in which the stent 20 has a cylindrical cross-sectional profile, the stent 20 may expand in diameter between about 1 mm and 3 mm beyond the compressed outer diameter of the stent 20 when the stent 20 is released from its compressed state. In other embodiments, the stent 20 may expand to a greater or lesser cross-sectional extent when it is released from the delivery catheter 36. The amount of expansion of the deployed self-expanding stent 20 will depend on the particular configuration of the stent 20 and other anatomical factors present during the surgical procedure.

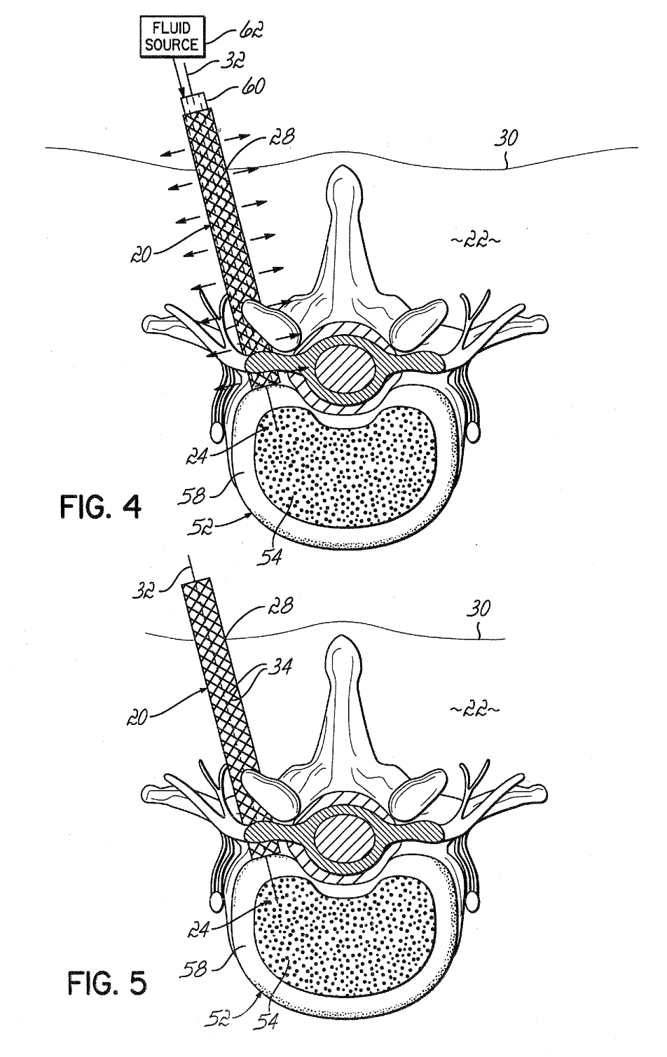

Referring now to FIGS. 3 and 4, following deployment of the stent 20 at the surgical site 24, a balloon 60 may be advanced along the guide wire 32 and inserted into the expanded stent 20. Alternatively, the balloon 60 may be inserted into the stent 20 without guidance along the guide wire 32. The balloon 60 is connected in a known manner to a fluid source 62 so that the balloon 60 may be expanded outwardly to thereby expand the stent 20 outwardly to a second cross-sectional extent greater than the first cross-sectional extent. The amount of expansion of the stent 20 will depend on the configuration of the stent 20, the configuration and operation of the inflation balloon 60 and the desired size and shape of the working channel 26 defined by the expanded stent 20. In one embodiment, in which the stent 20 has a cylindrical cross-sectional profile, the stent 20 may expand in diameter to about 24 mm, although other expanded diameters of the stent 20 are possible as well. The balloon 60 can be expanded to a desired atmospheric pressure so that the pressure can be kept below a level that causes tissue injury. Additionally, the inflation of the balloon 60, and hence the expansion of the stent 20, may be generally continuous and the rate of expansion controlled to minimize further tissue injury during the deployment of the stent 20.

As shown in FIG. 5, the balloon 60 is removed following expansion of the stent 20. The expanded stent 20 defines the working channel 26 extending from the point of entry 28 to the surgical site 24. The guide wire 32 may be left in place within the working channel 26 so that devices such as drills, reamers, screws, cages and the like can be advanced along the guide wire 32 to the surgical site 24. Following the surgical procedure, the stent 20 may be at least partially collapsed by grasping the proximal end of the stent 20 by hand or by instrument and withdrawing the stent 20 from the point of entry 28.

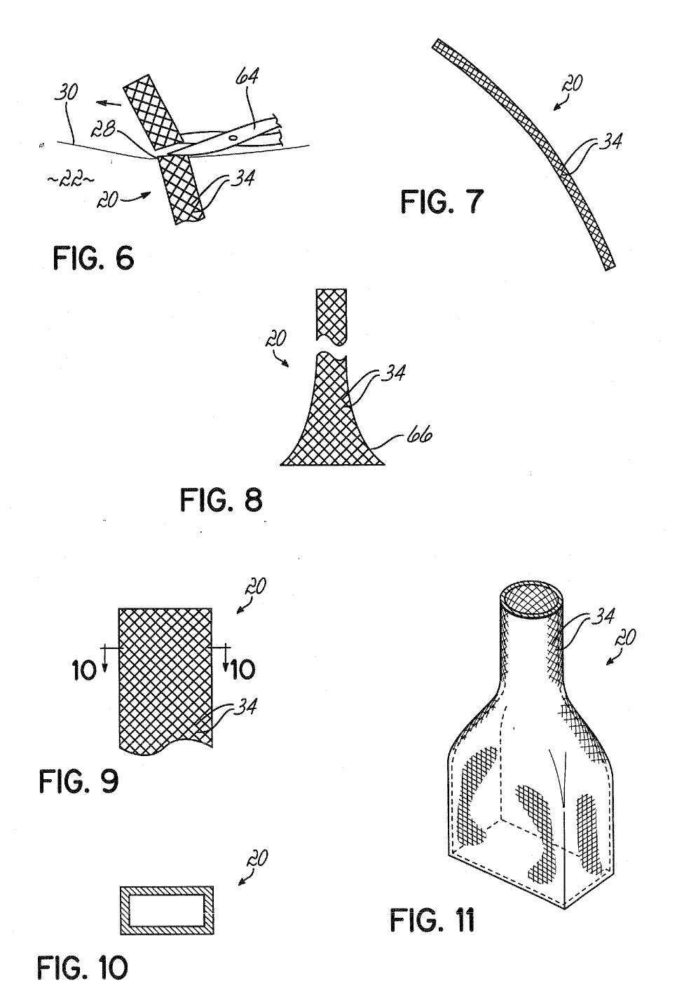

As shown in FIG. 6, excess stent material outside of the point of entry 28 can be easily trimmed away by the surgeon using a pair of scissors 64 or other suitable instrument so that the proximal end of the stent 20 may be generally flush with the patient's skin 30 if so desired by the surgeon. This shortens the surgeon's working distance and makes the stent 20 customizable to any surgical environment. In spinal surgical procedures, for example, the stent 20 may be used for cervical, thoracic or lumbar procedures. The variable outward expansion and variable length of the stent 20 reduces what has to be stocked by the hospital and reduces the need for pre-operative planning in terms of the desired length and diameter of the working channel 26.

In one embodiment, a first balloon is inserted into the deployed stent 20 to expand the stent 20 outwardly to a first cross-sectional extent greater than the outer diameter of the deployed, self-expanded stent 20. The first balloon is removed from the stent 20 and thereafter a second balloon is inserted into the stent 20 to expand the stent 20 outwardly to a second cross-sectional extent greater than the first cross-sectional extent. The second balloon is then removed from the expanded stent 20 so that the expanded stent 20 defines the desired size and shape of the working channel 26. For example, a surgeon can start at one diameter, such as about 12 mm, for a simple discectomy and then open the stent 20 outwardly a greater cross-sectional extent by using a larger balloon. This allows the surgeon to increase the exposure of the surgical site for the same case or for more complex procedures that need to be performed on the patient following the discectomy.

In another embodiment, a first stent may be deployed along a first guide wire and expanded at a first surgical site, such as the boney structures of the spine. Thereafter, the first guide wire may be removed and a second guide wire may be inserted through the first stent to a second surgical site, such as into the intervertebral disc space. Then, a second stent may be deployed in similar fashion via the second guide wire and expanded in a telescoping manner in relation to the first stent so the distance from the bone and the disc is now spanned.

As shown in FIG. 7, the stent 20 may be constructed to be flexible along at least a portion of its length to accommodate a curved trajectory to the surgical site and/or manipulation of the stent 20 by a surgeon during a procedure. In its deployed and expanded state, the stent 20 may define a working channel 26 that is curved along at least a partial length of the stent 20. The expanded cross-sectional profile of the stent 20 need not be cylindrical. For example, as shown in FIG. 8, a distal end 66 of the stent 20 may be flared when a flared balloon (not shown) is used to expand the stent 20 outwardly. In another embodiment as shown in FIGS. 9 and 10, the cross-sectional profile of the stent 20 may be generally rectangular as the result of a correspondingly shaped balloon used to expand the stent 20. Moreover, the stent 20 may be formed with more than one cross-sectional profile along its length as shown in FIG. 11. In this embodiment, the stent 20 may have a generally cylindrical cross-sectional profile along part of its length that transitions into a generally rectangular cross-sectional profile along a different part of its length. The curvature and various cross-sectional shapes obtainable with the stent 20 of the present invention are variable and selectable by the surgeon depending on the configuration of the stent 20, the requirements for a particular surgical procedure and the configuration of the balloon(s) used to expand the stent outwardly.

While the invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicants' general inventive concept.

What is claimed is:

1. A surgical instrument for providing access to a surgical site from a point of entry, comprising:
a stent body configurable from a collapsed first configuration to an expanded second configuration, the stent body further being operable, in the expanded second configuration, to provide access to the surgical site through a working channel defined by the stent body between the point of entry and the surgical site.

2. The surgical instrument of claim 1, wherein the stent body is generally a self-expanding stent body.

3. The surgical instrument of claim 1, wherein the stent body is generally flexible along at least a portion of its length.

4. The surgical instrument of claim 3, wherein the stent body defines a working channel that is non-linear along at least a partial length of the stent body.

5. The surgical instrument of claim 1, wherein the stent body is an expandable integral component.

6. The surgical instrument of claim 1, further comprising a membrane operatively connected to the stent body.

7. The surgical instrument of claim 1, further comprising an illumination system associated with the stent body and operable to illuminate the working channel.

8. The surgical instrument of claim 1, wherein the stent body comprises a mesh structure.

9. The surgical instrument of claim 1, further comprising a bioactive material associated with the stent body.

10. A surgical instrument for providing access to a surgical site from a point of entry, comprising:
a generally self-expanding stent body configurable from a collapsed first configuration to an expanded second configuration, the stent body further being operable, in the expanded second configuration, to provide access to the surgical site through a working channel defined by the stent body between the point of entry and the surgical site; and
an illumination system associated with the stent body and operable to illuminate the working channel.

11. The surgical instrument of claim 10, wherein the stent body is generally flexible along at least a portion of its length.

12. The surgical instrument of claim 11, wherein the stent body defines a working channel that is non-linear along at least a partial length of the stent body.

13. The surgical instrument of claim 10, wherein the stent body is an expandable integral component.

14. The surgical instrument of claim 11, further comprising a membrane operatively connected to the stent body.

15. The surgical instrument of claim 10, wherein the stent body comprises a mesh structure.

16. The surgical instrument of claim 10, further comprising a bioactive material associated with the stent body.

17. A method for providing access to a surgical site from a point of entry using an expandable stent, comprising:
guiding the stent from the point of entry to the surgical site; and
expanding the stent outwardly to provide access to the surgical site through a working channel defined by the expanded stent between the point of entry and the surgical site.

18. The method of claim 17, wherein the expanding step comprises:
positioning a balloon within the stent; and
expanding the balloon outwardly at the surgical site to expand the stent.

19. The method of claim 18, further comprising the steps of:
guiding the balloon to the surgical site;
positioning the balloon within the stent at the surgical site; and
expanding the balloon outwardly at the surgical site to expand the stent.

20. The method of claim 17, further comprising the step of illuminating the working channel.

21. The method of claim 17, wherein the expanding step comprises:
expanding the stent outwardly to a first cross-sectional extent; and
thereafter expanding the stent outwardly to a greater second cross-sectional extent.

22. The method of claim 21, further comprising the steps of:
positioning a first balloon within the stent;
expanding the first balloon outwardly at the surgical site to expand the stent to the first cross-sectional extent;
removing the first balloon from the stent;
positioning a second balloon within the stent; and
expanding the second balloon outwardly at the surgical site to expand the stent to the second cross-sectional extent.

23. The method of claim 17, further comprising the step of bending the stent along at least a portion of its length.

24. The method of claim 17, further comprising the steps of:
providing a first cross-sectional profile to the expanded stent at a first portion thereof; and
providing a second cross-sectional profile to the expanded stent at a second portion thereof.

25. The method of claim 17, further comprising the step of varying the length of the stent

26. The method of claim 17, wherein the stent is a generally self-expanding stent.

27. The method of claim 17, wherein the stent is generally flexible along at least a portion of its length.

28. The method of claim 27, wherein the stent defines a working channel that is non-linear along at least a partial length of the stent.

29. The method of claim 17, wherein the stent is an expandable integral component.

30. The method of claim 17, wherein the guiding step is performed with a guide wire.

31. A method for providing access to a surgical site from a point of entry using a generally self-expandable stent, comprising:
collapsing the stent;
guiding the collapsed stent from the point of entry to the surgical site; and
expanding the stent outwardly to provide access to the surgical site through a working channel defined by the expanded stent between the point of entry and the surgical site.

32. The method of claim 31, wherein the expanding step comprises releasing the stent from its collapsed state.

33. The method of claim 32, wherein the expanding step further comprises:
positioning a balloon within the released stent; and
expanding the balloon outwardly at the surgical site to expand the released stent.

34. The method of claim 33, further comprising the steps of:
guiding the balloon to the surgical site;
positioning the balloon within the released stent at the surgical site; and
expanding the balloon outwardly at the surgical site to expand the released stent.

35. The method of claim 31, wherein the stent is generally flexible along at least a portion of its length.

36. The method of claim 35, wherein the stent defines a working channel that is non-linear along at least a partial length of the stent.

37. The method of claim 31, wherein the stent is an expandable integral component.

38. The method of claim 31, wherein the guiding step is performed with a guide wire.
